(19) United States
(12) Plant Patent Application Publication
(10) Pub. No.: US 2004/0006799 P1
(43) Pub. Date: Jan. 8, 2004

(54) 'HW616' Pear

(76) Inventors: David Michael Hunter, St. Catharines (CA); Frank Kappel, Summerland (CA); Harvey Allen Quamme, Penticton (CA); William Gordon Bonn, Windsor (CA)

Correspondence Address: FLYNN, THIEL, BOUTELL & TANIS, P.C., 2026 Rambling Road, Kalamazoo, MI, 49008-1699, US

(21) Appl. No.: 10/190,392

(22) Filed: Jul. 3, 2002

(51) Int. Cl. A01H 5/00

(52) U.S. Cl. PLT/176

(57) ABSTRACT

‘HW616’ is an early season high quality pear primarily for the fresh market that has an attractive golden yellow fruit with no blush, smooth skin, very good balance between sweetness and acidity, that is exceptionally juicy and has excellent resistance to natural fire blight.

### FIELD OF THE INVENTION

The present invention relates to a pear (Pyrus communis L.) variety and more specifically to a pear variety that is a high-quality, early season pear for the fresh market.

### BACKGROUND OF THE INVENTION

The variety ‘HW616’ was developed by Agriculture and Agri-Food Canada at Harrow, Ontario. Plant Breeders rights was applied for in Canada, Application number 00-2185, filed Mar. 28, 2000. This application is still pending. Plant Breeders Rights were applied for in France on Jan. 18, 1995 and were granted as Certificate No. 012247 on Jun. 21,2001. The variety has also been described by Hunter et al, HortScience, Vol. 37 (1):224-226, February 2002. To applicants' knowledge, the variety has not been made publicly available. It will be marketed under the brand name AC HARROW GOLD.

### SUMMARY OF THE INVENTION

The new and distinct pear variety, which has been given the designation of ‘HW616’, produces a high quality early season pear primarily for the fresh market. ‘HW616’ is picked about ten days before ‘Bartlett’, the reference cultivar it most closely resembles. ‘HW616’ has an attractive golden yellow fruit with no blush, smooth skin, very good flavor with a good balance between sweetness and acidity, and exceptionally juicy. Fruit size of ‘HW616’ is equivalent to that of ‘Bartlett’. The tree or ‘HW616’ is medium in size, upright to spreading. ‘HW616’ has excellent resistance to natural fire blight (Erwina amylovora) infections (9.6 rating), whereas ‘Bartlett’ is susceptible to natural infections (4.2 rating). The response to infection following inoculation with the causative organism is more severe in ‘Bartlett’ (mean lesion length 63% of shoot length, with some lesions extending into subtending woody tissues) than in ‘HW616’ (mean lesion length 25% of shoot length, maximum lesion length of 52% of shoot length).

### BRIEF DESCRIPTION OF THE FIGURE

FIG. 1 is a photographic illustration of the whole fruit of ‘HW616’ pear.

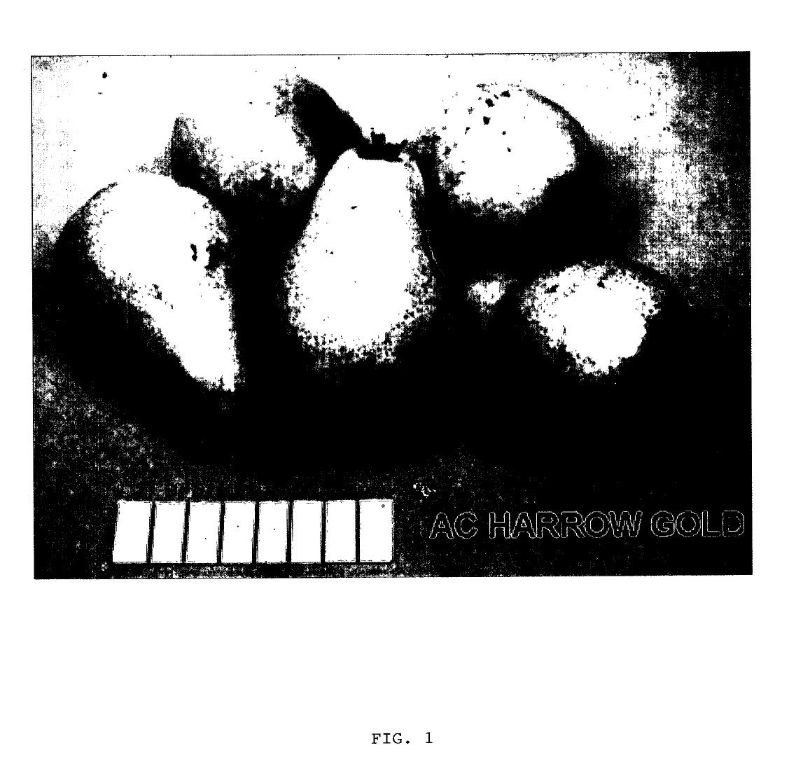

### DETAILED DESCRIPTION OF THE VARIETY

‘HW616’ resulted from a controlled cross between ‘Harvest Queen’ (patent status unknown) and ‘Harrow Delight’ (patent status unknown) made in 1975 by Dr. H. A. Quamme at the Agriculture and Agri-Food Canada Research Centre at Harrow, Ontario. It was selected as a hybrid seedling (H7535-100) in 1983 by F. Kappel, and propagated by budding onto pear seedling rootstocks. In 1985, it was advanced and testing began in 1987 at the Harrow Research Centre. Propagation for regional trials in cooperation with the Western Ontario Fruit Testing Association began in 1985, and test trees were placed in regional trials beginning in 1987. Evaluations of second test orchards have been conducted by F. Kappel and D. M. Hunter, and W. G. Bonn has evaluated disease resistance.

Asexual propagation maintains uniformity and stability of ‘HW616’. No variants, off-types or mutants have been observed. The variety will be maintained at the Agriculture and Agri-Food Canada Harrow Research Centre in a virus tested budwood orchard, and in the holdings of the Canadian Clonal Gene bank.

The color terminology is in accordance with The Royal Horticultural Society (RHS) Color Chart.

### Description and Performance: Tree Characteristics

Tree Habit and Productivity.

The tree of ‘AC Harrow Gold’ is medium in size, upright to spreading, annually productive and winter hardy. In 1994 at Harrow, following winter minimum temperatures as low a −29° C., a full crop was produced by the original seedling tree, as well as by trees grown on Bartlett seedling rootstock. The original seedling tree has consistently produced good crops with no evidence of biennial bearing. Precocity of ‘AC Harrow Gold’ trees propagated on Bartlett seedling rootstock appears to be similar to that of ‘Bartlett’, with bearing initiated ≈4 years after planting. Annual yields in the early years of production have been slightly lower than those of ‘Bartlett’, except when fire blight infections have reduced ‘Bartlett’ performance.

Shoot Habit.

The bark on the sun-exposed side of dormant shoots is light brown with olive (RHS 199A, Royal Horticultural Society, 1966). The diameter of dormant shoots of ‘AC Harrow Gold’ is similar to that of ‘Bartlett’ (data not presented). Mean internode length of ‘AC Harrow Gold’ was similar to ‘Bartlett’, ‘Harrow Delight’, and ‘Harrow Sweet’ (patented), but shorter than ‘Harvest Queen’ and ‘Bosc’ (Table 1).

TABLE 1
Mean internode length (cm ± SE) as
determined on five successive internodes
from the midportion of 1-year-old shoots.

| | Internode | No. of |
|---|---|---|
| Cultivar | length | Shoots |
| AC Harrow Gold | 3.0 ± 0.1 | 19 |
| Bartlett | 3,3 ± 0.1 | 13 |
| Harrow Delight | 3.1 ± 0.2 | 13 |
| Harrow Sweet | 3.1 ± 0.1 | 15 |
| Harvest Queen | 3.7 ± 0.2 | 17 |
| Bosc | 4.4 ± 0.1 | 14 |

^{z}Shoots harvested in Feb., 1996, from mature trees (>10 years old) grown on Bartlett seedling rootstock at Harrow, Ont., Canada.

Leaves.

The leaves are elliptic. The shape of the base of the leaf blade is right-angled. The shape of the upper part of the leaf blade is right-angled with a pointed acuminate tip. There is little curvature of the midrib. Leaf serrations are small and shallow but distinct. The angle between the petiole and the shoot is between 30° and 60°, the petiole is medium to long, and stipules are present. The attitude of the leaf in relation to the shoot is horizontal to slightly downward. Actively growing shoot tips are green with little pubescence.

Fire Blight Resistance.

As with other introductions from the Harrow pear breeding program (Hunter et al., 2002; Hunter et al., 1992; Quamme and Spearman, 1983), ‘AC Harrow Gold’ has excellent resistance to fire blight, similar to or greater than that of ‘Kieffer’ which is used as the standard for selection (Hunter, 1993). Using natural fire blight infection scores (van der Zwet et al., 1970), ‘AC Harrow Gold’ had a resistance rating similar to its parents, ‘Harrow Delight’ and ‘Harvest Queen’, and much greater than ‘Bartlett’ (Table 2). When actively growing shoot tips were inoculated-with a mixture of six virulent strains of E. amylovora, the length of the lesion that developed extended to 25% of current season's growth, similar to ‘Harrow Delight’, ‘Harvest Queen’, and ‘Kieffer’ (patent status unknown), but much less than lesion development in ‘Bartlett’ (Table 2).

Bloom and Pollination.

At Harrow, the time of full bloom of ‘AC Harrow Gold’ is similar to that of ‘Harrow Delight’ and ‘Harvest Queen’, and is ≈2 d earlier than ‘Bartlett’. First bloom, however, is 1 d later than ‘Bartlett’. Flower clusters typically contain six or seven flowers, occasionally five or eight. Petals are white, almost as long as broad, and touch or slightly overlap. The pink to red anthers are medium in size, and are about level with the stigma.

In controlled pollination tests, fruit set was used to determine pollen compatibility when pollen from a known source was applied to stigmatic surfaces immediately after emasculation of the flower. Because emasculated pear flowers are even less attractive to bees and other pollinating insects than non-emasculated flowers, bagging was not considered necessary. ‘AC Harrow Gold’ appears to be reciprocally pollen compatible with ‘Bartlett’, ‘Seckel’ (patent status unknown), ‘Old Home’ (patent status unknown), and ‘Harvest Queen’. While ‘AC Harrow Gold’ will pollinate ‘Harrow Delight’, ‘Harrow Delight’ will not pollinate ‘AC Harrow Gold’. Results of reciprocal pollinations between ‘AC Harrow Gold’ and ‘Bosc’ (patent status unknown) have been variable and inconclusive.

### Fruit Characteristics

Size, Shape and Color.

Fruits are medium sized, with a fruit length similar to ‘Harrow Delight’, longer than ‘Harvest Queen’, and slightly, but not significantly, smaller than ‘Bartlett’ (Table 3). Fruit diameter of ‘AC Harrow Gold’ was similar to ‘Harvest Queen’ and ‘Harrow Delight’ and significantly smaller than ‘Bartlett’. Fruit size and weight are improved by fruit thinning. Fruit shape is symmetrical, pyriform, and slightly concave to straight in profile. Using International Board for Plant Genetic Resources descriptors (Thibault et al., 1983), the predominant fruit shape has been described as 5.2 (≈25% of individual fruits) or 5.4 (≈25%), while other individual fruits have been described as 3.3 (≈10%), 3.4 (≈15%), 7.2 (≈10%), and 7.4 (≈10%). The calyx is persistent at harvest, with medium to long sepals that are convergent to upright. The calyx basin is medium depth and narrow to medium in width, with a slightly ribbed margin. Following ripening at ≈20° C., the skin has an attractive golden yellow ground color (RHS 11A or 11B) with no more than a trace of blush on the sun-exposed fruit surface, and there is little or no russetting. The flesh is cream to cream-white in color, is exceptionally juicy, and has excellent flavor with a good balance between sweetness and acidity.

Maturity.

At Harrow, Ont., Canada, the fruits of ‘AC Harrow Gold’ mature about 15 Aug., between ‘Harrow Delight’ and ‘Harvest Queen’, and ≈13 d before ‘Bartlett’. Unlike ‘Harrow Delight’, pre-harvest fruit drop is not a problem.

Quality.

At Harrow, fruits were harvested each year at the normal fresh market maturity for commercial harvest (5-7 kg pressure). Following ripening at ≈20° C. until ‘eating ripe’, a sample of 5 to 10 fruits, selected at random, was evaluated for appearance, flavor, texture, number and size of grit (stone cells) in the flesh, juiciness, and core size relative to fruit size. Evaluations were made on fruits ripened immediately after harvest. At Harrow, trained panelists rated the appearance of ripened fruits of ‘AC Harrow Gold’ equivalent to ‘Bartlett’, ‘Harrow Delight’ and ‘Harvest Queen’ (Table 3). The flesh texture was excellent, with little or no grit. The fresh fruit quality rating for ‘AC Harrow Gold’, as indicated by the weighted score, was similar to ‘Bartlett’, ‘Harrow Delight’ and ‘Harvest Queen’ (Table 3). There were no significant differences between fruits ripened immediately after harvest and fruits ripened after 4 weeks in a common cold storage at ≈2° C. (data not presented).

Processing Evaluations.

When ripened fruits are processed as halves or as puree, ‘AC Harrow Gold’ is rated similar to ‘Harrow Delight’ and ‘Harvest Queen’, but lower than ‘Bartlett’ (Table 4). While the processed product from small scale trials has been rated good, the quality may not be sufficiently high for ‘AC Harrow Gold’ to have commercial acceptability for processing in the current market. The commercial processing potential of this new cultivar will be further evaluated when the large scale plantings established in 1999 come into production.

Availability

‘AC Harrow Gold’ was tested at the Canadian Centre for Plant Health, Saanichton, B.C., using woody-host and herbaceous-host biological indicators, and by serological and molecular methods, and found to be free of all known viruses, virus-like agents, viroids and phytoplasmas. Virus-tested trees have been planted in the Canadian Clonal Gene Bank at Harrow.

TABLE 2
Ratings of natural and induced fire blight infections of
‘AC Harrow Gold’ in comparison to ‘Harrow Delight’,
‘Harvest Queen’, ‘Bartlett’, and ‘Kieffer’.

| | | | | |
|---|---|---|---|---|
| AC | | | | |
| Harrow | Harrow | Harvest | | |
| Gold | Delight | Queen | Bartlett | Kieffer |

Natural infections^{z}

| | | | | | |
|---|---|---|---|---|---|
| Rating | 9.6 ± | 9.5 ± | 9.1 ± | 4.2 ± | 9.3 ± |
| | 0.1 | 0.1 | 0.1 | 0.4 | 0.1 |
| Years | 10 | 19 | 20 | 18 | 15 |
| evaluated | | | | | |

Induced infections^{y}

| Lesion | 24.6 ± | 16.2 ± | 22.0 ± | 62.8 ± | 23.1 ± |
|---|---|---|---|---|---|
| (% shoot length) | 4.4 | 3.0 | 3.3 | 4.2 | 1.6 |
| Years | 10 | 12 | 12 | 22 | 16 |
| evaluated | | | | | |
| ^{z}Natura1 fire blight infections, rated on a scale of 1 (tree dead) to 10 (no blight), are means ± SE of 10 to 20 years at Harrow, Ont., Canada. The rating system was modified from van der Zwet et al. (1970) by assigning values of 10 = no visible blight and 9 = <3% infection. For ‘AC Harrow Gold’, ‘Harrow Delight’, and ‘Harvest Queen’, ratings were made on the own-rooted seedling tree. For ‘Bartlett’and ‘Kieffer’, ratings | | | | | |
| # were made on trees grown on Bartlett seedling rootstock in a nearby (within 200 m) cultivar evaluation orchard. In both the seedling orchard and the cultivar evaluation orchard, susceptible trees had severe fire blight infections each year. | | | | | |

^{y}Induced infections were measured in late July, ≈5 weeks after inoculating 10-20 actively growing shoots with 20 μL of a cocktail of six virulent strains of Erwinia amylovora (10⁸ cfu/mL). Values indicate lesion length expressed as a percentage of total shoot length. Data are means ± SE of 10 to 22 years.

TABLE 3
Harvest dates at Harrow, Ont., Canada, and fresh fruit
evaluations for ‘Harrow Delight’, ‘AC Harrow Gold’,
Harvest Queen’, and ‘Bartlett’

| | Harrow | AC Harrow | Harvest | |
|---|---|---|---|---|
| | Delight | Gold | Queen | Bartlett |
| Years | 22 | 12 | 21 | 20 |
| evaluated | | | | |
| | Harvest dates | | | |
| Average | 11 Aug. | 15 Aug. | 18 Aug. | 28 Aug. |
| Earliest | 27 Jul. | 31 Jul. | 6 Aug. | 18 Aug. |
| Latest | 18 Aug. | 22 Aug. | 24 Aug. | 9 Sep. |

| | Size (mm) | | | | | | | |
|---|---|---|---|---|---|---|---|---|
| Length | 80.4 | a^{z} | 80.5 | a | 72.4 | b | 81.4 | a |
| Diameter | 58.7 | b | 59.1 | b | 58.0 | b | 63.4 | a |
| | Ratings^{y} | | | | | | | |
| Appearance^{x} | 7.8 | a | 7.6 | a | 7.5 | a | 7.7 | a |
| Flavor^{x} | 8.2 | a | 8.0 | a | 8.3 | a | 8.0 | a |
| Texture^{x} | 8.0 | ab | 8.5 | a | 8.5 | a | 7.9 | b |
| Weighted | 82.5 | a | 83.6 | a | 82.9 | a | 81.3 | a |
| score^{w} | | | | | | | | |
| Grit^{v} | 4.1 | bc | 4.4 | ab | 4.6 | a | 3.9 | c |
| Juice^{u} | 4.4 | a | 4.1 | ab | 4.2 | ab | 3.9 | b |
| Core^{t} | 3.0 | a | 2.7 | b | 2.4 | b | 3.2 | a |

^{z}Means separation within rows by Duncan's new multiple range test, P = 0.05. Means within rows followed by the same letter are not significantly different.
^{y}Ratings reported are based on evaluations of fruits ripened immediately after harvest.
^{x}Appearance, flavor and texture ratings [on a scale of 1 (least desirable) to 9 (most desirable)] were determined each year by two to four trained panelists.
^{w}Weighted score = (3 × appearance) + (5 × flavor) + (2 × texture).
^{v}Grit rating is on a scale of 1 (undesirable, i.e. large and/or many grit cells) to 5 (desirable, i.e., very small and/or few or not grit cells).
^{u}Juiciness rating is on a scale of 1 (dry) to 5 (very juicy).
^{t}Core size rating is on a scale of 1 (small) to 5 (large).

TABLE 4
Ratings of pear halves^{z} and pear purée^{y} processed from
ripened fruits of ‘AC Harrow Gold’ in comparison with
‘Harrow Delight’, ‘Harvest Queen’, and ‘Bartlett’.

| | AC Harrow | Harrow | Harvest | |
|---|---|---|---|---|
| | Gold | Delight | Queen | Bartlett |
| Halves | 3.5 b^{x} | 3.4 b | 3.5 b | 3.9 a |
| Purée | 3.3 b | 3.2 b | 3.5 b | 4.0 a |

^{z}Determined with masked identity taste panels involving four trained panelists. Fruits were processed in syrup containing 15% (w/v) sugar. Processing rating for pear halves is the average of ratings for flavor, texture and appearance on a scale of 1 (least desirable) to 5 (most desirable). Samples, including a masked identity ‘Bartlett’ sample, were compared to a known ‘Bartlett’ sample. Data presented are means of 5 years.
^{y}Determined with masked identity taste panels involving four trained panelists. Fruits were processed with no additional sugar. Processing rating for purée is the average of ratings for viscosity, color, and flavor on a scale of 1 (least desirable) to 5 (most desirable). Samples, including a masked identity ‘Bartlett’ sample, were compared to a known ‘Bartlett’ sample. Data presented are means of 5 years.
^{x}Means separation within rows by Duncan's new multiple range test, P = 0.05. Means within rows followed by the same letter are not significantly different.

What is claimed is:

1. A new and distinct pear variety substantially as shown and described herein.
